(19) United States
(12) Patent Application Publication
(10) Pub. No.: US 2026/0265169 A1
(43) Pub. Date: Sep. 10, 2026

(54) METHOD FOR PRODUCING A MUSK FRAGRANCE INTERMEDIATE USING AlCl3 AS CATALYST

(71) Applicant: International Flavors & Fragrances Inc., New York, NY (US)

(72) Inventors: Sheng Wang, Zhejiang (CN); Feng Zheng, Zhejiang (CN)

(21) Appl. No.: 19/470,275

(22) PCT Filed: Mar. 27, 2024
(86) PCT No.: PCT/US2024/021713

§ 371 Date: Sep. 29, 2025

(30) Foreign Application Priority Data
Apr. 11, 2023 (CN) 202310383744.5

(51) Int. Cl. C07C 43/13; B01J 27/10; C07C 41/03

(52) U.S. Cl. CPC C07C 43/13; B01J 27/10; C07C 41/03

(57) ABSTRACT

Disclosed is a method for producing a musk fragrance intermediate by reacting an alcohol with an epoxide in the presence of AlCl₃ as catalyst.

### BACKGROUND

In the fragrance industry there is a constant demand for compounds having hedonic odor properties. Such compounds extend a perfumer's palette and result in greater product diversity for consumers. In particular, there is demand for compounds that have musk odor characteristics. Such compounds are highly esteemed in perfumery and are perhaps some of the most versatile and common compounds found in fragrance compositions. Exemplary musk fragrances include, e.g., Helvetolidee (Firmenich), Romandolide® (Firmenich), Serenolide (Givaudan), and Appelide (International Flavors & Fragrances Inc.) and derivatives thereof. These, and other musk fragrances are described in, e.g., WO 2002/096852 A1 to Givaudan SA; WO 2004/050595 A1 to Givaudan SA; WO 2004/050602 A1 to Givaudan SA; WO 2005/108534 A1 to Givaudan SA; WO 2011/29895 A2 to Givaudan SA; U.S. Pat. No. 5,166,412 A to Firmenich SA; WO 2000/014051 A1 to Firmenich SA; WO 2009/034510 A2 to Firmenich SA; U.S. Pat. No. 6,384,269 B1 to Firmenich SA; WO 2005/01222 2 Al and EP 1492759 B1 to Symrise AG; US 2004/053811 A1 to International Flavors & Fragrances Inc.; and WO 2019/124533 A1 to Takasago International Corp.

Given the value of these musk fragrances, needed in the art is a cost effective, high yield method for producing said compounds. The present invention addresses this need in the art.

### SUMMARY OF THE INVENTION

This invention provides a method for producing a musk fragrance intermediate (e.g., a musk fragrance intermediate of Formula (I), in particular demol) by reacting an alcohol (e.g., an alcohol of Formula (II), in particular cyclademol) with an epoxide (e.g., an epoxide of Formula (III), in particular isobutylene oxide) in the presence of AlCl₃ as catalyst thereby producing the musk fragrance intermediate. In some aspects, the method is carried out at a reaction temperature in the range of 30-35° C. and/or is carried out at atmospheric pressure.

### DETAILED DESCRIPTION OF THE INVENTION

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are used to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. For example, when a range of “1 to 10” is recited, the recited range should be construed as including ranges “1 to 8,” “3 to 10,” “2 to 7,” “1.5 to 6,” “3.4 to 7.8,” “1 to 2 and 7-10,” “2 to 4 and 6 to 9,” “1 to 3.6 and 7.2 to 8.9,” “1-5 and 10,” “2 and 8 to 10,” “1.5-4 and 8,” and the like.

The present disclosure illustratively described herein suitably may be practiced in the absence of any element or elements, limitation or limitations that are not specifically disclosed herein. While compositions and methods are described herein in terms of “comprising” various components or steps, the compositions and methods also can “consist essentially of” or “consist of” the various components or steps, unless stated otherwise.

A person of ordinary skill in the art appreciates that some chemical compounds in this disclosure have chiral center, carbon-carbon double bond, and/or cyclic structure. Unless explicitly indicated, a chemical compound in this disclosure includes its stereoisomers, such as enantiomers and diastereomers.

The musk fragrance Helvimor® is produced via a 2-step process from cyclademol (1-(3,3-dimethylcyclohexyl)ethanol) (Scheme 1). The key intermediate in this process is Demol (2-[1-(3,3-dimethylcyclohexyl)ethoxy]-2-methylpropan-1-ol), which has been synthesized from cyclademol using BF₃ (see EP 2200963 B1) or stoichiometric amounts of SnCl₂ as catalysts. Due to the use of high amounts of SnCl₂ (26 wt %), the yield per pass is very low (approximately 28 wt %). The present disclosure provides a simple, commercially feasible method for producing Demol using AlCl₃ as the catalyst. In addition to being inexpensive, this catalyst provides an increase in the selectivity of Demol compared to the use of SnCl₂.

Moreover, there is a significant reduction in the amount of solvent used, as well as waste-water produced.

Accordingly, this disclosure provides a method for producing a musk fragrance intermediate by reacting a suitable alcohol with an alkylating agent, in particular an epoxide, in the presence of AlCl₃ as catalyst thereby selectively achieving o-alkylation of the alcohol and producing the musk fragrance intermediate.

In particular aspects, this disclosure provides for the production of a musk fragrance intermediate of Formula (I):

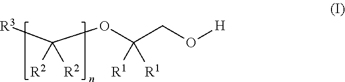

wherein
n represents 1 or 0,
each R¹, independently from each other, represents a hydrogen atom or a methyl or ethyl group, or the two R¹ taken together represent a (CH₂)ₘ group, m representing 3, 4, or 5;
each R², independently from each other, represents a hydrogen atom or a methyl or ethyl group, or the two R² taken together represent a (CH₂)ₘ group, m representing 3, 4, or 5; and
R³ represents a phenyl group optionally substituted, a saturated or unsaturated C₅-C₆ cyclic hydrocarbon moiety optionally substituted, or a CH(R⁴)₂, or R⁴CH═CR⁴ moiety, R⁴ representing a C₁-C₆ alkyl or alkenyl group optionally substituted.

According to particular aspects of this disclosure, the compound of Formula (1) is one in which n is 1, each R¹ is independently a hydrogen atom or methyl group, each R² is independently a hydrogen atom or methyl group, and R³ represents a phenyl group optionally substituted, a saturated or unsaturated C₅-C₆ cyclic hydrocarbon moiety optionally substituted, or a CH(R⁴)₂, or R⁴CH═CR⁴ moiety, R⁴ representing a C₁-C₆ alkyl or alkenyl group optionally substituted.

In some aspects, optional substituents of R³ are one, two or three C₁-C₃ alkyl groups, C₁-C₃ alkenyl groups, or C₁-C₃ alkoxy groups. In particular optional substituents of R³ are one, two or three methyl or ethyl groups. Non-limiting typical examples of R³ groups include 3,3-dimethyl-cyclohexyl, 3,3-dimethylcyclohex-1-en-1-yl, 4-methyl-pent-2-en-2-yl, 5-methyl-cyclohex-3-en-1-yl, and 2-methyl-cyclohexyl. In particular aspects, the musk fragrance intermediate of Formula (1) is demol (2-[1-(3,3-dimethylcyclohexyl)ethoxy]-2-methylpropan-1-ol).

In certain aspects, the alcohol used as the starting compound is an alcohol of Formula (II):

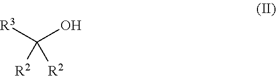

wherein each R², independently from each other, represents a hydrogen atom or a methyl or ethyl group, or the two R² taken together represent a (CH₂)ₘ group, m representing 3, 4, or 5; and
R³ represents a phenyl group optionally substituted, a saturated or unsaturated C₅-C₆ cyclic hydrocarbon moiety optionally substituted, or a CH(R⁴)₂, or R⁴CH═CR⁴ moiety, R⁴ representing a C₁-C₆ alkyl or alkenyl group optionally substituted. In particular aspects, the alcohol of Formula (II) is cyclademol (1-(3,3-dimethylcyclohexyl)ethanol).

Epoxides are cyclic ethers with three-membered cyclic rings composed of an oxygen atom attached to two adjacent carbon atoms. Epoxides of use in the method of this invention may have from 3 to 25 carbon atoms and one epoxy group. Exemplary epoxides include ethylene oxide, propylene oxide (1,2-propene oxide), butylene oxide (1,2-butene oxide), pentylene oxide (also known as 1,2-epoxypentane), hexylene oxide (also known as 1,2-epoxyhexane), octylene oxide (also known as 1,2-epoxyoctane), nonylene oxide (also known as 1,2-epoxynonane), decylene oxide (also known as 1,2-epoxydecane), isobutylene oxide, 4-methyl-1-pentylene oxide, and styrene oxide. In certain aspects, the epoxide used in the method of this invention is an epoxide of Formula (III).

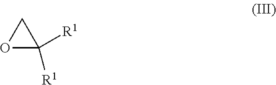

wherein each R¹, independently from each other, represents a hydrogen atom or a methyl or ethyl group, or the two R¹ taken together represent a (CH₂)ₘ group, m representing 3, 4, or 5. In particular aspects, the epoxide used in the process of the invention is isobutylene oxide.

In some aspects, the method of this disclosure is carried out in the presence of a catalyst, and the catalyst comprises, consists essentially of, or consists of AlCl₃. In some aspects, the AlCl₃ catalyst can be dry (e.g., in anhydrous form) or in hydrated form. In some aspects, the AlCl₃ catalyst is not loaded on a catalyst support (e.g., zeolite or activated carbon). In some aspects, the method of this disclosure is carried out in a reaction zone, and the AlCl₃ catalyst fed into the reaction zone is not in a form of a complex with a ligand. In some aspects, the catalyst is AlCl₃.

In some aspects, the reaction is carried out in the presence of a reduced amount of solvent (e.g., water and/or an organic solvent). Accordingly, in some aspects, a musk fragrance intermediate of Formula (I) is prepared by reacting an alcohol of Formula (II) with an epoxide of Formula (III) in the presence of no more than about 20%, 25%, 30%, 33%, or 35% solvent by weight of the reaction mixture (including starting materials, products and byproducts, and catalysts). In other aspects, demol is prepared by reacting cyclademol with isobutylene oxide in the presence of AlCl₃ in the presence of no more than about 20%, 25%, 30%, 33%, or 35% solvent by weight of the reaction mixture.

The reaction temperature at which a musk fragrance intermediate of Formula (I) is prepared, that is, the reaction temperature in the methods of this disclosure, is ideally between 20° C. and 40° C., or more preferably in the range of between 30° C. and 35° C. In a particular aspect, demol is prepared by reacting cyclademol with isobutylene oxide in the presence of AlCl₃ at a reaction temperature in the range of between 20° C. and 40° C., or more preferably in the range of between 30° C. and 35° C. Ideally the reaction is carried out at atmospheric pressure in batch or semi-batch mode. However, in some cases, the reaction may be carried out under reduced pressures conditions, e.g., between 0.5 and 100 mbar in batch mode or semi-batch mode.

In some aspects, the mole ratio (also expressed herein as equiv. or Eq.) of catalyst to alcohol (e.g., cyclademol) is in the range of 0.1 to 0.4, or more preferably in the range of 0.2 to 0.3. In other aspects, the mole ratio of epoxide (e.g., isobutylene oxide) to alcohol (e.g., cyclademol) is in the range of 0.5 to 2.0, or more preferably 0.6 to 1.0. In other aspects, the amount of catalyst is less than 20 wt % of the reaction mixture, less than 18 wt % of the reaction mixture, less than 15 wt % of the reaction mixture, less than 10 wt % of the reaction mixture, or less than 8 wt % of the reaction mixture.

The present method provides the advantage of overall improvements in process productivity, including a decrease in the reaction time and increase molar selectivity compared to SnCl₂. Moreover, in some aspects, the reaction uses less solvent than a comparable reaction with SnCl₂. As such, the present reaction provides for reduced wastewater production and manufacturing costs associated with the preparation of musk fragrance intermediates.

### Example 1: Conventional Process for Synthesizing Helvimor from Cyclademol Using SnCl₂(Scheme 1)

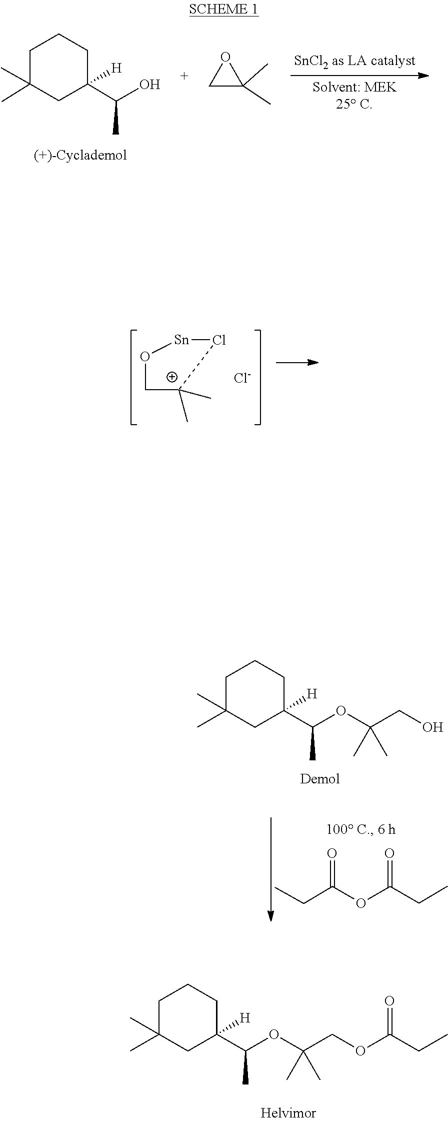

In general, cyclademol, catalyst and other reagents (solvent, internal standard, etc.) are loaded in the reactor. When the desired temperature is reached, isobutylene oxide addition starts. Samples are taken, analyzed by gas chromatography and when the reaction is finished, crude product is directly quenched with acid hydrolysis using diluted hydrochloric acid. The product is washed with water and neutralized with diluted sodium hydroxide. The organic crude product is distilled with a fractional column in order to recover unreacted cyclademol and Demol.

Demol and sodium hydroxide (0.11 wt %) as catalyst are loaded into a reactor. At 110° C., propionic anhydride (1.3 eq) addition begins. When addition is finished, the temperature is increased to 130° C. The reaction is finished when Demol <1% by gas chromatography. The crude product is directly quenched with sodium hydroxide solution (1.5 eq NaOH). The product is washed with water and hexane is used to facilitate the separation between the aqueous and organic phase. The final organic phase is distilled to obtain Helvimor©.

### Example 2: Screening of Catalysts

Various catalysts were tested for their ability to catalyze the conversion of cyclademol and isobutylene oxide to demol. For these reactions, cyclademol, catalyst and other reagents (solvent, internal standard, etc.) were loaded in the reactor (Table 1). When the desired temperature was reached, isobutylene oxide addition began (Table 2). Samples were periodically taken and analyzed by gas chromatography. When the reaction was finished, crude product was directly quenched with acid hydrolysis using diluted hydrochloric acid. The product was washed with water and neutralized with diluted sodium hydroxide. In these tests, temperature and addition time of isobutylene oxide (Table 2), reaction time and temperature (Table 2), catalyst (Table 1) and the amount of isobutylene oxide (in equivalence to cyclademol) (Table 2), and solvents (Table 1) were adjusted as needed. Based on, inter alia, percent conversion, selectivity and yield (Table 3), as well as ease of use, AlCl₃ was suitable substitute for SnCl₂ in the synthesis of demol.

TABLE 1

| | | Eq. | Solvent | |
|---|---|---|---|---|
| Ex. | Catalyst | Catalyst¹ | Type | wt. %² |
| 1 | SnCl₂ | 0.24 | MEK | 57 |
| 2 | ZnCl₂ | 0.15 | Dodecane | 25 |
| 3 | Cu(NO₃)₂ | 0.10 | Dodecane | 25 |
| 4 | FeCl₃ | 0.15 | Dodecane | 25 |
| 5 | BF₃ diethyl ethearate | 0.02 | — | — |
| 6 | Amberlyst 35 dry | 0.20 | — | — |
| 7 | CuCl | 0.15 | Dodecane | 25 |
| 8 | AlCl₃ | 0.23 | DCM | 33.7 |
| 9 | AlCl₃ | 0.23 | EB | 33.7 |
| 10 | ZnCl₂ | 0.23 | MEK | 33.7 |
| 11 | SnCl₂ | 0.26 | MEK | 25 |
| 12 | SnCl₄ | 0.26 | MEK | 25 |
| 13 | Bu₃SnOMe | 0.26 | MEK | 25 |
| 14 | BF₃•OEt₂ | 0.26 | MEK | 25 |
| 15 | Ti(Oct)₄ | 0.26 | MEK | 25 |
| 16 | SiO₂•SO₃ | 0.26 | MEK | 25 |
| 17 | FeCl₃ | 0.26 | MEK | 25 |
| 18 | Fe(NO₃)₃ | 0.26 | MEK | 25 |
| 19 | Fe₂(SO₄)₃ | 0.26 | MEK | 25 |
| 20 | Fe(acac)₃ | 0.26 | MEK | 25 |
| 21 | Fe₂O₃ | 0.26 | MEK | 25 |
| 22 | K₄FeCN₆ | 0.26 | MEK | 25 |
| 23 | CuCl | 0.26 | MEK | 25 |
| 24 | CuBr | 0.26 | MEK | 25 |
| 25 | CuCO₃ | 0.26 | MEK | 25 |
| 26 | CuCN | 0.26 | MEK | 25 |
| 27 | Cu(acetate)₂ | 0.26 | MEK | 25 |
| 28 | Cu(acac)₂ | 0.26 | MEK | 25 |
| 29 | Cu(SO₃CF₃)₂ | 0.26 | MEK | 25 |
| 30 | Cu(CO₂CF₃)₂ | 0.26 | MEK | 25 |
| 31 | CuSO₄ | 0.26 | MEK | 25 |
| 32 | Cu(NO₃)₂ | 0.26 | MEK | 25 |
| 33 | Cu(NO₃)₂ | 0.26 | MEK | 25 |
| 34 | CuO/SiO₂ (12 wt %) | 0.26 | MEK | 25 |
| 35 | NaX•CuBr₂ (5 wt %) | 0.26 | MEK | 25 |
| 36 | NaX•CuBr₂ (10 wt %) | 0.26 | MEK | 25 |
| 37 | Cu(NO₃)₂•12H₂O | 0.26 | MEK | 25 |
| 38 | CuO/SiO₂ (5 wt %) | 0.2 | — | — |

Ti(Oct)₄, Titanium tetraoctanate; X, zeolite X.
¹Eq. Catalyst, Catalyst equivalent to cyclademol (i.e., the mole ratio of catalyst to cyclademol).
²wt. %, wt % of solvent with respect the mixture of cyclademol + solvent.
DCM, dichloromethane. EB, ethylbenzene.

TABLE 2

| | T (° C.) | Addition | Reaction T | Reaction | Eq. |
|---|---|---|---|---|---|
| Ex. | Addition | time (h) | (° C.) | time (h) | IBO¹ |
| 1 | 25 | 7 | 35 | 23 | 0.84 |
| 2 | RT | 1 | 35 | 22 | 1.00 |
| 3 | RT | 1 | 35 | 24 | 1.00 |
| 4 | RT | 1 | 35 | 2 | 1.00 |
| 5 | 0-5 | 2 | 0 | 1.5 | 1.00 |
| 6 | 0-5 | 1 | 40 | 22 | 0.75 |
| 7 | RT | 1 | 35 | 45 | 1.00 |
| 8 | 5-15 | 1 | 35 | | 0.73 |
| 9 | 5-15 | 1 | 35 | | 0.73 |
| 10 | 5-15 | 1 | 35 | | 0.73 |
| 11 | 25 | 0.03 | 35 | 18 | 0.75 |
| 12 | 25 | 0.03 | 35 | 18 | 0.75 |
| 13 | 25 | 0.03 | 35 | 18 | 0.75 |
| 14 | 25 | 0.03 | 35 | 18 | 0.75 |
| 15 | 25 | 0.03 | 35 | 18 | 0.75 |
| 16 | 25 | 0.03 | 35 | 18 | 0.75 |
| 17 | 25 | 0.03 | 35 | 18 | 0.75 |
| 18 | 25 | 0.03 | 35 | 18 | 0.75 |
| 19 | 25 | 0.03 | 35 | 18 | 0.75 |
| 20 | 25 | 0.03 | 35 | 18 | 0.75 |
| 21 | 25 | 0.03 | 35 | 18 | 0.75 |
| 22 | 25 | 0.03 | 35 | 18 | 0.75 |
| 23 | 25 | 0.03 | 35 | 18 | 0.75 |
| 24 | 25 | 0.03 | 35 | 18 | 0.75 |
| 25 | 25 | 0.03 | 35 | 18 | 0.75 |
| 26 | 25 | 0.03 | 35 | 18 | 0.75 |
| 27 | 25 | 0.03 | 35 | 18 | 0.75 |
| 28 | 25 | 0.03 | 35 | 18 | 0.75 |
| 29 | 25 | 0.03 | 35 | 18 | 0.75 |
| 30 | 25 | 0.03 | 35 | 18 | 0.75 |
| 31 | 25 | 0.03 | 35 | 18 | 0.75 |
| 32 | 25 | 0.03 | 35 | 18 | 0.75 |
| 33 | 25 | 0.03 | 60 | 18 | 0.75 |
| 34 | 25 | 0.03 | 35 | 18 | 0.75 |
| 35 | 25 | 0.03 | 35 | 18 | 0.75 |
| 36 | 25 | 0.03 | 35 | 18 | 0.75 |
| 37 | 25 | 0.03 | 35 | 18 | 0.75 |
| 38 | 25 | 0.03 | 35 | 18 | 1.4 |

“T(° C.) addition” is the temperature at which isobutylene oxide was added to the reaction. “Addition time (h)” is the duration over which the IBO was added. “Reaction T(° C.)” is the temperature at which the reaction was allowed to proceed after isobutylene oxide was added. “Reaction time (h)” was the time in which the reaction proceeded and included the time over which the IBO was added. RT, room temperature.
¹Eq. IBO, Isobutylene Oxide equivalent to cyclademol (i.e., the mole ratio of isobutylene oxide to cyclademol).

TABLE 3

| | | % | % Molar | |
|---|---|---|---|---|
| | | Conversion | Selectivity | % Molar |
| Ex. | Catalyst | Cyclademol | Demol | Yield Demol |
| 1 | SnCl₂ | 28 | 82 | 23.0 |
| 2 | ZnCl₂ | 26 | 60 | 15.6 |
| 3 | Cu(NO₃)₂ | 2 | 94 | 1.9 |
| 4 | FeCl₃ | 37 | 53 | 19.6 |
| 5 | BF₃ diethyl ethearate | 17 | 95 | 16.2 |
| 6 | Amberlyst 35 dry | 10 | 22 | 2.2 |
| 7 | CuCl | 1 | 0 | 0.0 |
| 8 | AlCl₃ | 21.6 | 88.0 | 19.0 |
| 9 | AlCl₃ | 21.6 | 86.1 | 18.6 |
| 10 | ZnCl₂ | 13 | 86.3 | 11.2 |
| 11 | SnCl₂ | 37 | 22 | 8.1 |
| 12 | SnCl₄ | 16 | 12 | 1.9 |
| 13 | Bu₃SnOMe | 12 | 0 | 0.0 |
| 14 | BF₃•OEt₂ | 0 | 0 | 0.0 |
| 15 | Ti(Oct)₄ | 30 | 0 | 0.0 |
| 16 | SiO₂•SO₃ | 28 | 2 | 0.6 |
| 17 | FeCl₃ | 32 | 12 | 3.8 |
| 18 | Fe(NO₃)₃ | 57 | 11 | 6.3 |
| 19 | Fe₂(SO₄)₃ | 26 | 14 | 3.6 |
| 20 | Fe(acac)₃ | 0 | 0 | 0.0 |
| 21 | Fe₂O₃ | 3 | 1 | 0.0 |
| 22 | K₄FeCN₆ | 24 | 0 | 0.0 |
| 23 | CuCl | 4 | 1 | 0.0 |
| 24 | CuBr | 8 | 4 | 0.3 |
| 25 | CuCO₃ | 0 | 0 | 0.0 |
| 26 | CuCN | 0 | 0 | 0.0 |
| 27 | Cu(acetate)₂ | 0 | 0 | 0.0 |
| 28 | Cu(acac)₂ | 0 | 0 | 0.0 |
| 29 | Cu(SO₃CF₃)₂ | 26 | 22 | 5.7 |
| 30 | Cu(CO₂CF₃)₂ | 42 | 22 | 9.2 |
| 31 | CuSO₄ | 0 | 0 | 0.0 |
| 32 | Cu(NO₃)₂ | 25 | 17 | 4.3 |
| 33 | Cu(NO₃)₂ | 32 | 13 | 4.2 |
| 34 | CuO/SiO₂ (12 wt %) | 13 | 10 | 1.3 |
| 35 | NaX•CuBr₂ (5 wt %) | 3 | 1 | 0.0 |
| 36 | NaX•CuBr₂ (10 wt %) | 4 | 2 | 0.1 |
| 37 | Cu(NO₃)₂•12H₂O | 19 | 6 | 1.1 |
| 38 | CuO/SiO₂(5 wt %) | 11 | 9 | 1.0 |

Ti(Oct)₄, Titanium tetraoctanate; X, zeolite X.

1. A method for producing a musk fragrance intermediate comprising reacting an alcohol with an epoxide in the presence of AlCl₃ as catalyst thereby producing a musk fragrance intermediate.

2. The method of claim 1, wherein the musk fragrance intermediate has the structure of Formula (I): 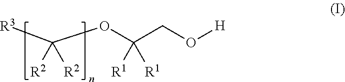
wherein
n represents 1 or 0,
each R¹, independently from each other, represents a hydrogen atom or a methyl or ethyl group, or the two R¹ taken together represent a (CH₂)ₘ group, wherein m represents 3, 4, or 5;
each R², independently from each other, represents a hydrogen atom or a methyl or ethyl group, or the two R² taken together represent a (CH₂)ₘ group, wherein m represents 3, 4, or 5; and
R³ represents a phenyl group optionally substituted, a saturated or unsaturated C₅-C₆ cyclic hydrocarbon moiety optionally substituted, a CH(R⁴)₂ moiety, or R⁴CH═CR⁴ moiety, wherein R⁴ represents a C₁-C₆ alkyl or alkenyl group optionally substituted.

3. The method of claim 1, wherein the alcohol has the structure of Formula (II): 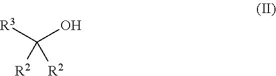
wherein
each R², independently from each other, represents a hydrogen atom or a methyl or ethyl group, or the two R² taken together represent a (CH₂)ₘ group, wherein m represents 3, 4, or 5; and
R³ represents a phenyl group optionally substituted, a saturated or unsaturated C₅-C₆ cyclic hydrocarbon moiety optionally substituted, a CH(R⁴)₂ moiety, or R⁴CH═CR⁴ moiety, wherein R⁴ represents a C₁-C₆ alkyl or alkenyl group optionally substituted.

4. The method of claim 1, wherein the epoxide has the structure of Formula (III): 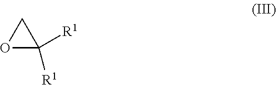
wherein
each R¹, independently from each other, represents a hydrogen atom or a methyl or ethyl group, or the two R¹ taken together represent a (CH₂)ₘ group, wherein m represents 3, 4, or 5.

5. The method of claim 1, wherein the musk fragrance intermediate is demol.

6. The method of claim 1, wherein the alcohol is cyclademol.

7. The method of claim 1, wherein the epoxide is isobutylene oxide.

8. The method of claim 1, wherein said method is carried out at a reaction temperature in the range of 30-35° C.

9. The method of claim 1, wherein said method is carried out at atmospheric pressure.
